(19) United States
(12) Patent Application Publication
(10) Pub. No.: US 2025/0027047 A1
(43) Pub. Date: Jan. 23, 2025

(54) CELL OBTAINING METHOD FOR CREATING NFT BASED ON CELL IMAGE

(71) Applicant: INDUSTRY-ACADEMIC COOPERATION FOUNDATION, YONSEI UNIVERSITY, Seoul (KR)

(72) Inventor: Jinkee HONG, Seoul (KR)

(73) Assignee: INDUSTRY-ACADEMIC COOPERATION FOUNDATION, YONSEI UNIVERSITY, Seoul (KR)

(21) Appl. No.: 18/714,704

(22) PCT Filed: Nov. 8, 2022
(86) PCT No.: PCT/KR2022/017488

§ 371 Date: Jul. 30, 2024

(30) Foreign Application Priority Data
Nov. 9, 2021 (KR) 10-2021-0153238
Nov. 7, 2022 (KR) 10-2022-0147345

(51) Int. Cl. C12N 5/077; C12N 5/0793

(52) U.S. Cl. CPC C12N 5/0657; C12N 5/0619; C12N 2506/25

(57) ABSTRACT

The present disclosure relates to a cell obtaining method for creating NFT based on a cell image. The cell obtaining method includes the steps of separating a cell from a biological sample, obtaining a stem cell by treating the cell with a dedifferentiation inducer, differentiating the stem cell, acquiring an image of the differentiated cell; and converting the image of the differentiated cell into an NFT image, and creates the NFT using a vital image in a simple and easy way through non-invasive and non-medical procedures.

### CROSS-REFERENCE TO RELATED APPLICATIONS

This application claims priority to and the benefit of Korean Patent Application No. 10-2021-0153238, filed Nov. 9, 2021, and Korean Patent Application No. 10-2022-0147345, filed Nov. 7, 2022, which are incorporated herein by reference in their entirety.

### TECHNICAL FIELD

The present disclosure relates to a cell obtaining method for creating NFT based on a cell image.

### BACKGROUND

As a blockchain technology market expands around the world, a non fungible token (NFT)-related technology is rapidly growing to implement proof of ownership not only for digital art or digital fashion items for virtual world avatars, but also for various goods in the real world.

The NFT is a type of digital registration certificate stored on the blockchain. The NFT includes the Internet address where the original is stored, owner identity information, and smart contract information, and is used as a means to buy and sell ownership of digital assets. The blockchain technology may be used to track a person who owns the NFT, and satisfy those who have a desire to own digital assets because counterfeiting and duplication of digital assets is impossible.

Until now, the NFT have been used as a means to satisfy people's desire to show off by purchasing the NFT for investment purposes in collectibles such as artwork and stamps or by registering the NFT as a profile on SNS, used as an admission ticket to participate in a project, community, or performance, or used for purposes such as applying the NFT to an authenticity authentication system in connection with physical products.

In addition, the NFT technology is currently being used usefully in the trading of game items or digital art, but the usefulness is not permanently guaranteed because the game items become useless when the related game services end. In addition, since the digital art can be completely duplicated, the ownership of the original file of the digital art has limited utility other than personal satisfaction, and since there are many cases where there is a separate copyright holder, the owner of digital art may have significant restrictions on its use in such cases.

In this way, the digital assets that the NFT may demonstrate their potential are still unclear and impermanent. Therefore, the development of various services is necessary so that the NFT may have greater influence with guaranteed functions or values.

Accordingly, there have been attempts to turn vital information into the NFT. Genomic big data such as DNA and blockchain convergence technology have the advantage of establishing a virtuous cycle in which all related industries, such as the public, research institutes, and drug developers, coexist. However, there are concerns about infringement on human dignity or individual privacy, such as personal information leakage and data management issues. In particular, the DNA is sensitive personal information and includes the information not only on the person involved but also on their family, and therefore, is still limitations in expecting active distribution and transactions as the NFT.

### SUMMARY

The present disclosure relates to a technology related to a vital image without a risk of problems that may result from leakage of personal information, and an object of the present disclosure provides a cell obtaining method for creating NFT based on a cell image for converting the cell image into NFT in a simple and easy way.

Another object of the present disclosure is to own a vital image of humans or animals, etc., own the vital image of precious family members or companion animals, or create and sell images created by collecting cells over time like a biography, or diversify a business model of digital assets such as combining the vital image with other products, by converting cell images derived from living organisms into digital assets based on creating NFT based on the cell images.

In one general aspect, the present disclosure may provide a cell obtaining method for creating NFT based on a cell image, including: (S100) separating a cell from a biological sample; (S200) obtaining a stem cell by treating the cell with a dedifferentiation inducer; (S300) differentiating the stem cell; (S400) acquiring an image of the differentiated cell; and (S500) converting the image of the differentiated cell into an NFT image.

The biological sample may be derived from oral tissue or urine.

The step (S100) may include performing first centrifugation on the biological sample, removing a supernatant, suspending cell sediment in primary medium, and then performing second centrifugation.

The step (S200) may include inoculating a cell into a gelatin-coated culture dish; and replacing a mixed medium with a mesenchymal stem cell medium.

The dedifferentiation inducer may be one or more selected from Oct3/4, Sox2, Klf4, L-Myc, and Lin28a.

In step (S400), the cell image may be acquired as an imaging result using a fluorescence microscopy, a confocal microscopy, a phase-contrast microscopy, an electron microscopy, bright field imaging, or a combination thereof.

In step (S400), the cell image may be an image with sound added.

In step (S500), the NFT image may be a combination of the cell image and digital content.

According to the present disclosure, it is possible to easily acquire cell images in a non-invasive and non-medical procedure, and to create NFT that can be used for multiple purposes by combining the NFT with other digital content.

In addition, according to the present disclosure, it is possible to create and sell vital images, such as humans or animals, as NFTs, to create and sell images created by collecting cells over time like a biography, or to provide various business models such as combining the vital images with other products.

### BRIEF DESCRIPTION OF THE DRAWINGS

FIG. 1 is a flowchart illustrating the overall process of a cell obtaining method for creating NFT based on a cell image according to the present disclosure.

FIG. 2 is a schematic diagram illustrating a process of acquiring stem cells (UDC-derived iPSCs) by treating a urine-derived cell with a dedifferentiation-inducing factor, according to an embodiment of the present disclosure.

FIG. 3 is a diagram illustrating a microscopic image of cells observed during subculture of the urine-derived cells according to an embodiment of the present disclosure. After cell inoculation, 13 days have passed (passage 0) and 15 days have passed (passage 1).

FIG. 4 is a schematic diagram illustrating a differentiation process of urine-derived stem cells into cardiomyocytes according to an embodiment of the present disclosure.

FIG. 5 is a diagram illustrating a microscopic image observing the differentiation process of the urine-derived stem cells over time, according to an embodiment of the present disclosure.

FIG. 6 is a diagram illustrating results of confirming an expression of Brachyury and MIXL1 through electrophoresis during the differentiation process of the urine-derived stem cells according to an embodiment of the present disclosure.

FIG. 7 is a diagram illustrating the results of confirming an expression of Ryr2, Myh6, and cTnT through electrophoresis during the differentiation process of the urine-derived stem cells according to an embodiment of the present disclosure.

FIG. 8 is a diagram illustrating the results of acquiring cell images differentiated into cardiac muscle cells and nerve cells from the urine-derived stem cells according to an embodiment of the present disclosure.

FIG. 9 is an example diagram illustrating a configuration of an NFT including a nerve cell image according to an embodiment of the present disclosure.

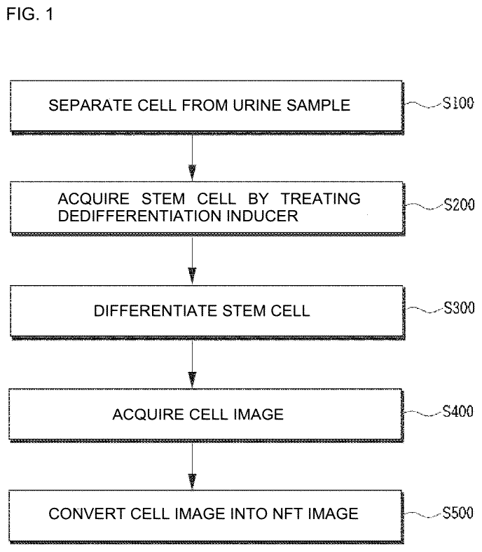

### DETAILED DESCRIPTION OF THE EMBODIMENTS

Hereinafter, a management system for generating and distributing a cell image-based NFT and a service providing method thereof according to the present disclosure will be described in detail with reference to the attached drawings so that a person skilled in the art can easily perform it.

Technical terms and scientific terms used in the present specification have the general meaning understood by those skilled in the art to which the present disclosure pertains unless otherwise defined, and a description for the known function and configuration unnecessarily obscuring the gist of the present disclosure will be omitted in the following description and the accompanying drawings.

The present disclosure may be implemented in various different forms and is not limited to embodiments or drawings described herein. In the drawings, portions unrelated to the description will be omitted to clearly describe the present disclosure, and similar portions will be denoted by similar reference numerals throughout the specification. The drawings are provided as examples to enable the idea of the present disclosure to be sufficiently conveyed to those skilled in the art to which the present disclosure pertains, the present disclosure is not limited to the presented drawings and may be embodied in other forms, and the drawings may be exaggerated to clarify the spirit of the present disclosure.

The singular forms used in the specification are intended to include the plural forms as well, unless the context specifically dictates otherwise.

In addition, “Including” mentioned herein is an open-ended description having an equivalent meaning to expressions such as “comprising,” “containing,” “having,” “characterizing,” and elements, materials, or processes not listed additionally are not excluded.

As used herein, the term “stem cell” is a cell that may differentiate into various cells that make up biological tissues, and refers to undifferentiated cells that may regenerate without limitation to form specialized cells of tissues and organs. The stem cell may divide into two daughter stem cells or one daughter stem cell and one transition cell, which may then proliferate into mature, complete cells of the tissue. The stem cell may be broadly divided into an embryonic stem cell, a somatic cell nuclear transfer, an adult stem cell, and an induced pluripotent stem cell (iPSCs). In the case of the embryonic stem cell, there are ethical issues with its use due to the need to use eggs, and in the case of the adult stem cell, it is difficult to obtain large amounts of undifferentiated cells due to limitations in in vitro proliferation. Therefore, in the present disclosure, the stem cells are preferably iPSCs.

In this specification, “suspension culture” refers to culturing cells to be cultured in a suspending state in a culture medium without cells to be cultured being fixed to a substrate, etc. The mesodermal stem cells, which are attachment-dependent, cause cell aggregation during the suspension culture, and cells that are not included in this aggregation and suspend alone may die by inducing apoptosis.

The NFT are a non-fungible token, and may serve as a solution to the problem of infinite duplication of digital information online. A value of uniqueness and scarcity is assigned, and specific virtual assets are recorded through blockchain, which is the basis of cryptocurrency. This is one of the basic characteristics of the blockchain and may be achieved through ‘distributed ledger technology’ that distributes blocks to users participating in a network. This has the advantage of being able to permanently maintain the original record without change, and provides a method for owning ‘digital assets’ that can be traded online, such as trading digital art and contracting real estate in the virtual world.

Due to its non-fungible nature, the NFT is currently being used in various fields such as art, sports, and fashion. The present disclosure was created through research to expand the utility of the NFT, and starts from converting vital images into the NFT. However, the vital information is an issue directly related to human dignity and sensitive personal information, and in the case of personal information recorded in the blockchain, it is difficult to delete personal information recorded on the blockchain and there is a risk of personal information being leaked due to poor data management. Accordingly, the present disclosure seeks to provide a method for safely and easily converting vital images into digital assets by creating NFT based on a cell image.

The present disclosure relates to a cell obtaining method for creating NFT based on a cell image, and the method may include: (S100) separating a cell from a biological sample; (S200) obtaining a stem cell by treating the cell with a dedifferentiation inducer; (S300) differentiating the stem cell; (S400) acquiring an image of the differentiated cell; and (S500) converting the image of the differentiated cell into an NFT image.

The biological sample refers to sample materials derived from living organisms from which cells can be collected, and may include blood, plasma, serum, lymph, saliva, urine, tissue lysate, culture fluid, semen, sweat, bone marrow fluid, eye fluid, brain extract, spinal fluid, synovial fluid, thymic fluid, ascites, amniotic fluid, and any other biological solution containing cells.

In the present disclosure, the biological sample may be oral tissue, hair, or urine, and as a specific example, cell separation may be performed from urine that can collected in non-invasive and non-medical procedures.

Since dermal fibroblasts are secured by punching a skin tissue, pain is involved for a donor, and since endothelial progenitor cells (EPCs) are collected from blood samples, medical procedures are inevitably involved. Also, in this case, the yield of pluripotent stem cells may decrease due to low reprogramming efficiency. On the other hand, urine derived cells (UDCs) can be easily proliferated and provide the advantage of not having to be collected repeatedly.

As an example, step (S100) may be separating cells from a urine sample. Specifically, it may include performing first centrifugation on the biological sample, removing a supernatant, suspending a cell sediment in primary medium, and then performing second centrifugation. The centrifugation may be performed at a temperature ranging from 200×g to 600×g for 5 to 20 minutes. After the first centrifugation, the settled cell sediment is suspended in the primary medium, and then after the second centrifugation, the settled cell sediment is suspended again in the primary medium. Thereafter, cells are inoculated into a gelatin-coated culture dish.

As a more specific example, 150 ml of human urine is centrifuged at 400×g for 10 minutes, the supernatant is discarded, the settled cells are suspended in primary medium (PM), and then are centrifuged again at 400×g for 10 minutes. The supernatant is discarded, and the settled cells are suspended again with 1 mL of PM, and are inoculated into a gelatin-coated 12-well plate culture dish. Thereafter, 1 mL of PM is newly replaced once a day and the cells are cultured for 3 days. On the third day of culture, the medium is replaced with a medium prepared by mixing 50% of PM and 50% of mesenchymal cell proliferation medium (MCPM) (hereinafter referred to as REMC). Thereafter, REMC is replaced and cultured every two days, and when 80% confluency is reached, cells are detached using trypsin-EDTA, transferred to a new gelatin-coated culture dish, and continues to be cultured to obtain cells.

The composition of the primary medium PM and the mesenchymal cell proliferation medium (MCPM) is as follows.

TABLE 1
Primary medium (PM) medium composition
Primary medium (PM)

DMEM/F12 (Thermo Fisher Scientific, Cat# 11320033)
10% Fetal Bovine Serum (FBS, Welgene, 5001-01)
1 kit REGM ™ SingleQuots ™ Kit (for 500 mL PM)
100 μg/mL Primocin ® (Invivogen, Cat# ant-pm-05)

TABLE 2
Mesenchymal cell proliferation medium (MCPM) medium composition
Mesenchymal cell proliferation medium(MCPM)

DMEM high glucose (Thermo Fisher Scientific, Cat# 11965092)
10% Fetal Bovine Serum (FBS, Welgene, 5001-01)
1× MEM, GlutaMax ™ (Thermo Fisher Scientific, Cat# 41090093)
1× MEM, Non-Essential Amina Acids (Thermo Fisher Scientific, Cat# 11140076)
5 ng/mL basic fibroblast growth factor (bFGF, Peprotech, CAT# AF-100-188)
5 ng/mL platelet-derived growth factor(PDGF)-AB (Peprotech, Cat# 100-00AB)
5 ng/mL epidermal growth factor(EGF) (Peprotech, Cat# AF-100-15)

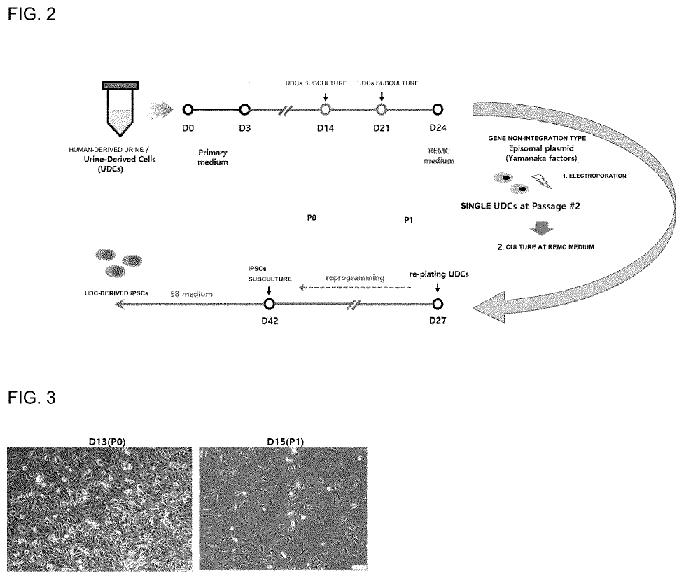

Referring to FIG. 2, after inoculating cells, the basic medium is replaced once a day and cultured for 10 days or more, 20 days or more, or 40 days or more to obtain stem cells. During this process, two or more subcultures may be performed. The urine-derived cells have the advantage of being easy to subculture several times.

In step (S200), reprogramming may be performed by treating the urine-derived cells with a dedifferentiation inducer. In this case, the dedifferentiation inducer is one or more selected from Oct3/4, Sox2, Klf4, L-Myc, and Lin28a.

The introduction of the inducer is possible through a viral vector or non-viral vector. As the viral vectors, for example, retrovirus, wrench virus, and adenovirus may be used. For example, using a non-integrating episomal plasmid as a non-viral vector reduces the risk of damage to genetic information and enables stable expression. However, it is not limited thereto, and gene introduction is possible according to known techniques.

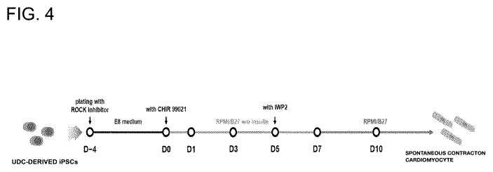

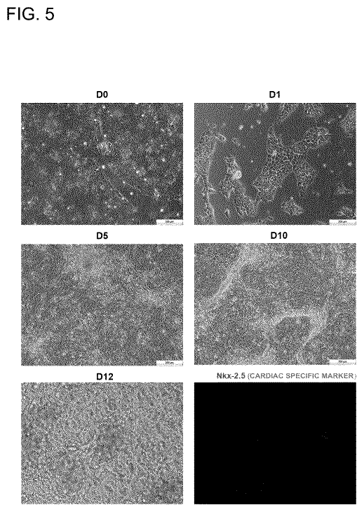

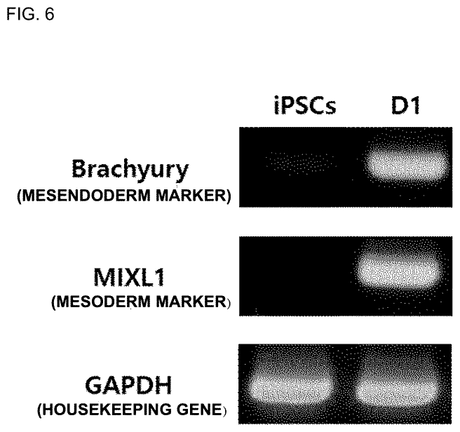

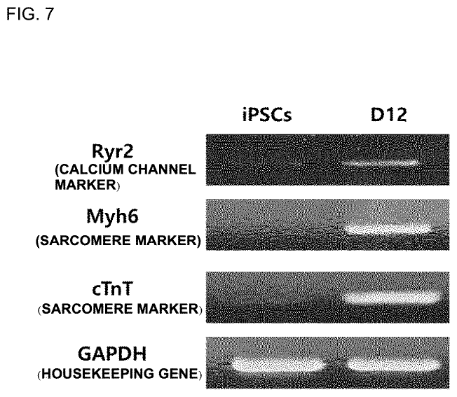

Referring to FIG. 4, step (S300) may include a pretreatment step of adding a ROCK inhibitor (Rho-associated kinase inhibitor). Apoptosis of stem cells may be inhibited by treatment with ROCK inhibitors. In this case, the stem cells may differentiate in 5 to 15 days, 10 to 15 days, or 12 to 15 days to form cardiomyocytes. FIG. 5 illustrates the differentiation process of stem cells, and it can be observed that differentiation into cardiomyocytes was successfully achieved through a heart-specific marker (Nkx-2.5) on day 12. In addition, referring to FIG. 7, it can be observed that the differentiation into cardiomyocytes was successfully achieved on day 12 through calcium channel marker Ryr2 and sarcomere marker Myh6 and cTnT.

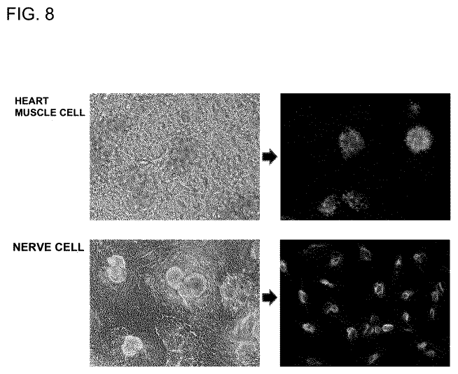

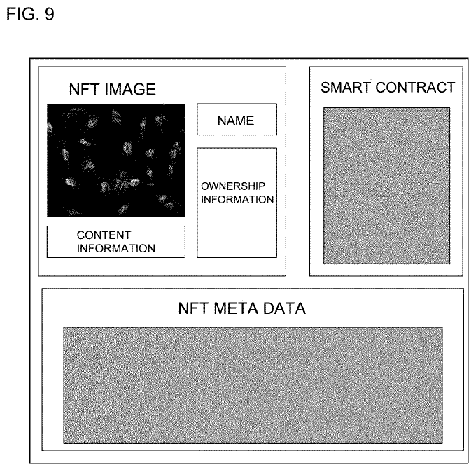

In step (S400), the cell image is acquired as an imaging result using a fluorescence microscopy, a confocal microscopy, a phase-contrast microscopy, an electron microscopy, bright field imaging, or a combination thereof. As illustrated in FIGS. 8 and 9, the urine-derived stem cells may be differentiated into cardiac muscle cells and nerve cells, converted into cell images, and converted into NFT.

However, this is just an example, and cell images may be obtained from all cells such as cardiac muscle tissue, nerve cell bundles, and vascular cell tubes, and may include all proliferation, differentiated states, etc.

The cell images may be not only static images, but also dynamic data that may confirm cell motions, cell movement, etc. In addition, the cell image may be a static image or dynamic data with sound added. As a specific example, when the cell is a cardiac muscle cell, the cell image may be dynamic data in which movement such as a heartbeat is recognized, and/or may be a static image or dynamic data to which a heartbeat sound is added.

The cell image may be an image obtained from cells derived from endangered animals or plants. By converting the image into the NFT and providing it, it may contribute to spreading awareness to preserve life at the risk of extinction from threats such as environmental pollution, and may be used to improve the profits of public projects.

The cell image may be used alone, and may be created in a fused form with other digital images, sounds, and other digital effects other than cells. The cell image may include meta data, which may include descriptions of the images, sounds, and other digital effects, production processes, and vital information. This information may be stored in any suitable form.

In the present disclosure, the NFT image may be a combination of a cell image and other works. Specifically, the digital content such as paintings or music may be converted into an NFT image by combining with the cell image. As a non-limiting example of the present disclosure, the NFT image may be a combination of cell images and other digital images. In addition, other analysis information on life (genome information, metabolic information, etc.) may be added and stored in the form of meta data.

In the present disclosure, the NFT may be used in combination with a product including a display unit. As a specific example, by registering an NFT converted from an image of a horse's heart muscle cell on a car instrument panel display, an image of a NFTied horse's heart muscle cell may be displayed on the display at the same time as a car is started. Therefore, when purchasing a product, customizing services may be provided to an NFT image licensee or owner. This can be the creation of a personalized product that contains my vital information, which is only one in the world, and if various technologies develop, it may also be used for digital identification purposes.

In addition, the cell image-based NFT according to the present disclosure may be used in various ways on metaverse platforms, and the scope of use may be determined depending on users. For example, it may be used as a profile picture on SNS, as a game item, as applying to an avatar, and as authentication or authorization.

In relation to embodiments of the present disclosure, the cryptocurrency used for the NFT conversion and transaction may be Ethereum, Polygon, self-issued coins, tokens, or the like, but is not necessarily limited thereto.

What has been described above is only an example for carrying out the cell obtaining method for cell image-based NFT production according to the present disclosure, and is not limited thereto. As claimed in the claims below, it is said that the technical idea of the present disclosure is to the extent that various modifications can be made by anyone skilled in the art without departing from the gist of the present disclosure.

1. A cell obtaining method for creating NFT based on a cell image, comprising:
separating a cell from a biological sample;
obtaining a stem cell by treating the cell with a dedifferentiation inducer;
differentiating the stem cell;
acquiring an image of the differentiated cell; and
converting the image of the differentiated cell into an NFT image.

2. The cell obtaining method of claim 1, wherein the biological sample is derived from oral tissue or urine.

3. The cell obtaining method of claim 1, wherein the step includes performing first centrifugation on the biological sample, removing a supernatant, suspending a cell sediment in primary medium, and then performing second centrifugation.

4. The cell obtaining method of claim 1, wherein the step includes inoculating a cell into a gelatin-coated culture dish; and replacing a mixed medium with a mesenchymal stem cell medium.

5. The cell obtaining method of claim 1, wherein the dedifferentiation inducer is one or more selected from Oct3/4, Sox2, Klf4, L-Myc, and Lin28a.

6. The cell obtaining method of claim 1, wherein in step, the cell image is acquired as an imaging result using a fluorescence microscopy, a confocal microscopy, a phase-contrast microscopy, an electron microscopy, bright field imaging, or a combination thereof.

7. The cell obtaining method of claim 1, wherein in step, the cell image is an image with sound added.

8. The cell obtaining method of claim 1, wherein in step, the NFT image is a combination of the cell image and digital content.
